(19) United States
(12) Patent Application Publication
(10) Pub. No.: US 2021/0322217 A1
(43) Pub. Date: Oct. 21, 2021

(54) OCULAR DRAINAGE SYSTEM DEVICES AND METHODS

(71) Applicant: W. L. Gore & Associates, Inc., Newark, DE (US)

(72) Inventors: Peter J. Roeber, Oxford, PA (US); Jeffrey C. Towler, Wilmington, DE (US)

(21) Appl. No.: 17/272,545

(22) PCT Filed: Aug. 29, 2019
(86) PCT No.: PCT/US2019/048760

§ 371 Date: Mar. 1, 2021

Related U.S. Application Data
(60) Provisional application No. 62/724,180, filed on Aug. 29, 2018.

(51) Int. Cl. A61F 9/007; A61L 27/16

(52) U.S. Cl. CPC A61F 9/00781; A61L 27/16

(57) ABSTRACT

Ocular drainage systems are disclosed. In various embodiments, the ocular drainage systems include a compliant fluid conduit that is configured to be implanted within a biological tissue, such as tissues of an eye. The compliant fluid conduit includes an exterior having a microstructure that is configured to permit cellular ingrowth. A first end of the compliant fluid conduit is configured to be inserted into an eye of a patient to allow ocular fluid to drain from the eye, and a second end of the compliant fluid conduit is configured to be inserted into an ocular venous system of the patient to allow the ocular fluid drained from the eye to flow directly into the ocular venous system.

### CROSS-REFERENCE TO RELATED APPLICATION

This application is a national phase application of PCT Application No. PCT/US2019/048760, internationally filed on Aug. 29, 2019, which claims the benefit of Provisional Application No. 62/724,180, filed Aug. 29, 2018, which are incorporated herein by reference in their entireties for all purposes.

### BACKGROUND

Aqueous humor is a fluid that fills the anterior chamber of the eye and contributes to the intraocular pressure or fluid pressure inside the eye. Glaucoma is a progressive disease of the eye characterized by an increase of the eye's intraocular pressure. This increase in intraocular pressure is commonly caused by a sufficient amount of aqueous humor not being reabsorbed by the body. In some cases, the aqueous humor is not absorbed fast enough or at all, while in others, the aqueous humor is additionally or alternatively being produced too quickly. An increase in intraocular pressure is associated with a gradual and sometimes permanent loss of vision in the afflicted eye.

A number of attempts have been made to treat glaucoma, including surgical procedures that implant a device that operates to drain some of the aqueous humor from the anterior chamber and dissipate the same to eye tissue for reabsorption. However, the implantation of conventional glaucoma drainage system devices has been associated with a number of complications including erosion and gradual decrease of absorption of the aqueous humor through the surrounding tissue due to scar tissue formation.

Additional complications are the result of the designs of the conventional devices, which typically lack the flexibility, conformity, and device/tissue attachment required to avoid micro-movement between the device and the surrounding tissue. This micro-movement sometimes leads to micro-irritations of the surrounding tissue, which is known to lead to a foreign body tissue response and excessive scar formation and thus a decrease in absorption performance of the surrounding tissue. In some cases, persistent micro-irritations of the surrounding tissue may lead to eventual erosion of the device and site infection, which is associated with gradual and sometimes permanent loss of vision in the afflicted eye. In instances where erosion does not occur, the scar tissue effectively prevents reabsorption of the aqueous humor otherwise evacuated from the anterior chamber by these conventional devices. These and other complications can serve to circumvent any beneficial therapy provided by conventional devices. The resulting effect is a gradual increase in intraocular pressure and glaucoma.

### SUMMARY

According to one example (“Example 1”), a medical device includes a compliant fluid conduit configured for implantation within a biological tissue (e.g., within or amongst tissues of the eye), the compliant fluid conduit having a first end, a second end, a lumen, and an exterior having a microstructure that is configured to permit cellular ingrowth therein; where the first end is configured to be inserted into an eye (e.g., into a chamber of the eye or near a chamber of the eye) of a patient to allow ocular fluid (e.g., aqueous humor) to drain from the eye; and where the second end is configured to be inserted into an ocular venous system of the patient to allow the ocular fluid drained from the eye to flow directly into the ocular venous system.

According to another example (“Example 2”), further to Example 1, a luminal wall surface of the lumen is configured to resist cellular ingrowth and attachment.

According to another example (“Example 3”), further to Example 2, the luminal wall surface of the lumen includes a plurality of pores sized to resist cellular ingrowth and attachment.

According to another example (“Example 4”), further to Example 2, the luminal wall surface of the lumen includes a microstructure that is configured to resist cellular ingrowth and attachment.

According to another example (“Example 5”), further to any of the preceding examples, the compliant fluid conduit is a polymer tube.

According to another example (“Example 6”), further to Example 5, the polymer tube includes a plurality of layers.

According to another example (“Example 7”), further to Example 6, the plurality of layers include a first layer having a first micro-structure and a second layer having a second micro-structure.

According to another example (“Example 8”), further to Example 5, the polymer tube includes a fluoropolymer.

According to another example (“Example 9”), further to Example 8, the polymer tube includes expanded polytetrafluoroethylene.

According to another example (“Example 10”), further to any of the preceding Examples, the medical device operates to regulate an intraocular pressure of a patient's eye when implanted.

According to another example (“Example 11”), further to any of the preceding Examples, the compliant fluid conduit is configured to allow fluid egress from within an anterior chamber of a patient's eye when implanted.

According to another example (“Example 12”), further to any of the preceding Examples, the compliant fluid conduit includes one of a plurality of lumens formed in a tubular structure, or a plurality of individual tubular elements, each tubular element including a lumen extending therethrough.

According to another example (“Example 13”), further to any of the preceding Examples, the medical device further includes a valve configured to regulate a rate of fluid flowing through the compliant fluid conduit.

According to another example (“Example 14”), further to Example 13, the valve is formed from partially unbonded helical windings of a material forming the compliant fluid conduit, where the valve is configured to regulate a rate of fluid backflowing through the compliant fluid conduit in a direction toward an anterior chamber of the eye.

According to another example (“Example 15”), further to Example 14, the valve is integral to the compliant fluid conduit such that the valve and the compliant fluid conduit form a monolithic unit.

According to another example (“Example 16”), further to any of the preceding Examples, the exterior of the compliant fluid conduit includes a plurality of pores sized to permit cellular ingrowth.

According to another example (“Example 17”), further to Example 16, the interior of the compliant fluid conduit includes a microstructure that is configured to resist cellular ingrowth and attachment.

According to another example (“Example 18”), further to any of the preceding Examples, the medical device further includes a sheath disposed about the compliant fluid conduit, the sheath defining the exterior of the compliant fluid conduit.

According to another example (“Example 19”), further to any of the preceding Examples, the second end of the compliant fluid conduit is configured to be inserted into an episcleral vein of the eye.

According to another example (“Example 20”), further to any of the preceding Examples, the compliant fluid conduit is a synthetic polymeric material that is nonbioabsorbable.

According to another example (“Example 21”), a method of treating glaucoma includes providing a compliant fluid conduit having a first end configured for insertion into an eye of a patient (e.g., into a chamber of the eye or near a chamber of the eye) and a second end configured for insertion into an ocular venous system of the patient, the compliant fluid conduit being configured for implantation within a biological tissue (e.g., within or amongst tissues of the eye) and including an exterior that is configured to permit cellular ingrowth therein; inserting the first end into the eye of the patient such that the first end of the compliant fluid conduit accesses a fluid reservoir within the eye; and inserting the second end of the compliant fluid conduit into the ocular venous system such that a fluid within the fluid reservoir within the eye is free to drain through the compliant fluid conduit into the ocular venous system.

According to another example (“Example 22”), further to Example 21, inserting the first end of the compliant fluid conduit into the eye of the patient includes inserting the first end into an anterior chamber of the eye of the patient.

According to another example (“Example 23”), further to Example 21, inserting the second end of the compliant fluid conduit into the venous system includes inserting the second end into an episcleral vein of the eye.

According to another example (“Example 24”), further to any of Examples 21 to 23, the compliant fluid conduit is a synthetic polymeric material that is nonbioabsorbable.

### BRIEF DESCRIPTION OF THE DRAWINGS

The accompanying drawings are included to provide a further understanding of inventive embodiments of the disclosure and are incorporated in and constitute a part of this specification, illustrate examples, and together with the description serve to explain inventive principles of the disclosure.

FIG. 1 is a schematic illustration of an ocular drainage system extending between an anterior chamber access of the eye and an episcleral vein access according to some embodiments.

FIG. 2A is a schematic illustration of an ocular drainage system implanted within an eye according to some embodiments.

FIG. 2B is a schematic illustration of an ocular drainage system implanted within an eye according to some embodiments.

FIG. 3 is a sectioned view of an eye showing an implanted ocular drainage system extending between an anterior chamber access of the eye and an episcleral vein access according to some embodiments.

FIG. 4 is a schematic illustration of an ocular drainage system including a soft, compliant tubular element according to some embodiments.

FIG. 5 is a schematic illustration of a second ocular drainage system including a soft, compliant tubular element according to some embodiments.

FIG. 6A is a schematic illustration of a normal flow operation through a tubular element of an ocular drainage system according to some embodiments.

FIG. 6B is a schematic illustration of an abnormal flow operation through a tubular element of an ocular drainage system according to some embodiments.

### DETAILED DESCRIPTION

Persons skilled in the art will readily appreciate that the various embodiments of the inventive concepts provided in the present disclosure can be realized by any number of methods and apparatuses configured to perform the intended functions. It should also be noted that the accompanying figures referred to herein are not necessarily drawn to scale, but may be exaggerated to illustrate various aspects of the present disclosure, and in that regard, the figures should not be construed as limiting.

The present disclosure is directed toward ocular drainage systems, devices, and methods for draining an ocular fluid from a fluid-filled chamber of a patient's eye so that it may be reabsorbed by the body. Providing a mechanism for reabsorption of ocular fluid that has been evacuated from a chamber of the eye operates to lower or otherwise stabilize the intraocular pressure. In particular embodiments, the ocular drainage systems, devices, and methods provide for draining aqueous humor (an ocular fluid) from the anterior chamber of a patient's eye.

In various embodiments, unlike conventional designs, the ocular drainage systems of the present disclosure are constructed of biologically inert, biocompatible, non-bioabsorbable synthetic materials that are configured to permit and/or promote the ingrowth of tissue into one or more regions of the microstructure of the ocular drainage system. Permitting such ingrowth helps minimize micro-movements between the ocular drainage systems and the surrounding tissue overtime. Minimization of micro- movements helps minimize irritation and micro-irritation of the surrounding tissue caused by interactions of the surrounding tissue and the ocular drainage system, and thus helps minimize a risk that the ocular drainage system will erode from the anatomy over time. As described below, the ocular drainage system provides a fluid pathway between a chamber of the eye (e.g., the anterior chamber of the eye and/or the posterior chamber of the eye) and one or more veins in the episcleral vasculature.

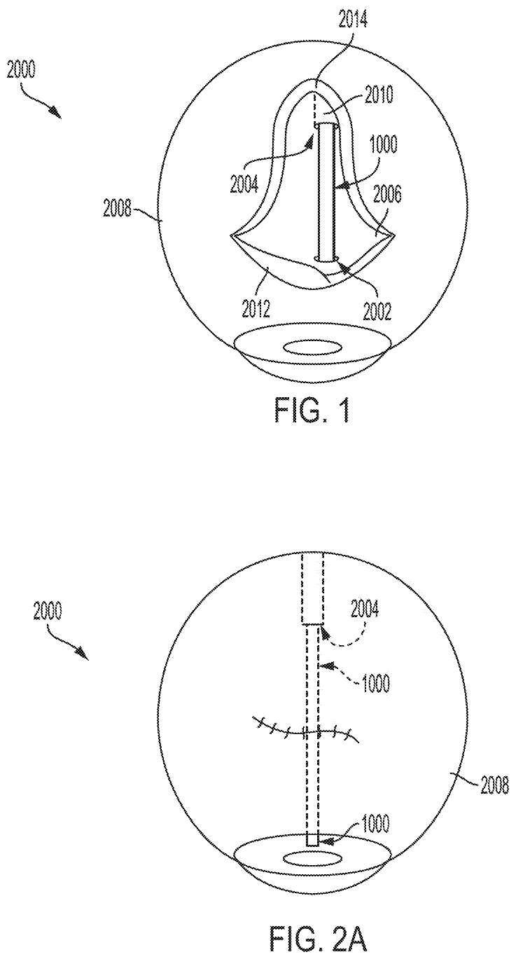

An ocular drainage system 1000 is illustrated in FIGS. 1 to 3 in association with an eye 2000. FIG. 1 shows the ocular drainage system 1000 extending between an anterior chamber access 2002 of the eye 2000 and an episcleral vein access 2004, although the ocular drainage system 1000 may similarly extend between the posterior chamber of the eye and the episcleral vein. For simplicity, the anterior chamber will be further described throughout the disclosure, although it will be recognized that the posterior chamber or the vitreous chamber may similarly be accessed and drained by the ocular drainage system 1000 if desired.

The anterior chamber (AC, FIG. 3) may be accessed by forming an incision, perforation, hole, or other access through the sclera 2006 of the eye 2000, such as at the limbus of the eye 2000. In some embodiments, one or more episcleral veins 2010 may be accessed by forming an incision, perforation, hole, or other access through the sclera 2006 of the eye, proximate the episcleral vasculature. Generally, the sclera 2006 is accessed via forming an incision through the conjunctiva 2008. In some embodiments, a pocket can be subsequently formed in a subconjunctival space between the conjunctiva 2008 and the sclera 2006 of the eye 2000. The pocket may be formed to provide space for accommodating the ocular drainage system 1000 within the anatomy (e.g., the eye). As used herein, the term “within a biological tissue” refers to placement within, between, and/or amongst tissue. In some embodiments, “within a biological tissue” refers to placement between two distinct tissues of an organ, such as, for example, between the conjunctiva and sclera of the eye.

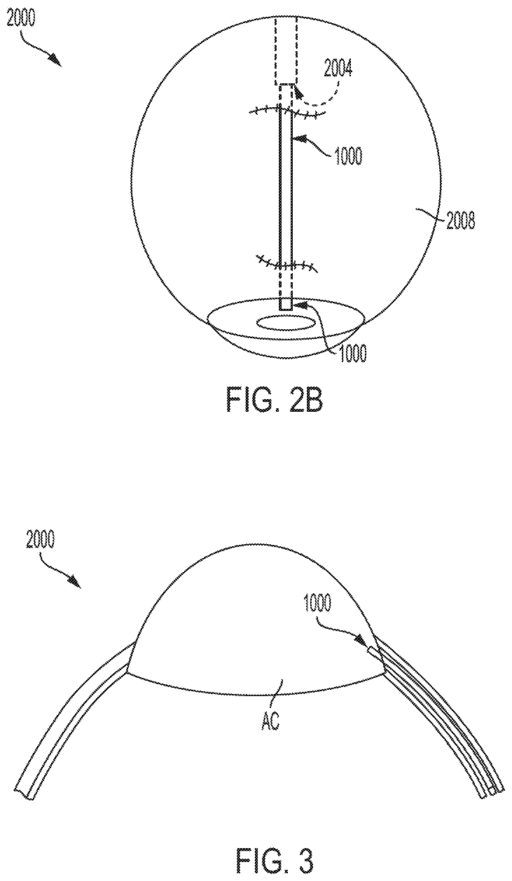

In FIG. 1, conjunctival flaps 2012 and 2014 are pulled aside to reveal a subconjunctival space between the conjunctiva 2008 and the sclera 2006 of the eye 2000. In FIG. 2A, the conjunctival flaps 2012 and 2014 are sutured closed such that the ocular drainage system 1000 extends between the anterior chamber access 2002 of the eye 2000 and the episcleral vein access 2004 beneath the conjunctiva 2008. While the examples discussed herein include the ocular drainage system 1000 that extends between the subconjunctival anterior chamber access 2002 and the subconjunctival episcleral vein access 2004 (e.g., the ocular drainage system 1000 is entirely beneath the conjunctiva 2008 and within the subconjunctival space), it is to be appreciated that the ocular drainage system 1000 may access one or more of the episcleral veins.

As illustrated in FIG. 2B, in some embodiments, the anterior chamber and the episcleral vasculature are accessed via separate incisions, perforations, holes, or other access means. A first incision or other access means occurs near the anterior chamber, while a second incision or access means occurs near and proximate the episcleral vasculature. In such configurations, a portion of the ocular drainage system 1000 remains on the exterior of the eye.

FIG. 3 illustrates an ocular drainage system 1000 implanted within an eye as depicted in FIG. 2A, where the ocular drainage system 1000 extends between the anterior chamber access 2002 of the eye 2000 and the episcleral vein access 2004 beneath the conjunctiva 2008.

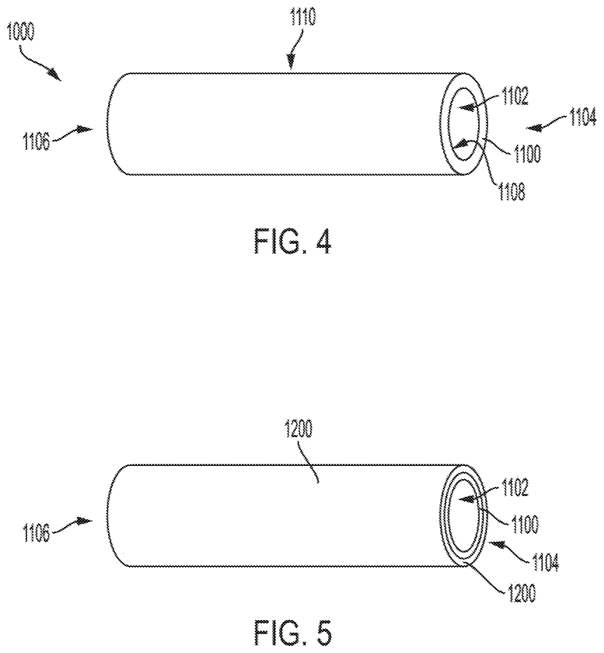

In some embodiments, the ocular drainage system 1000 includes a soft and compliant tubular element 1100. As shown in FIG. 4, the tubular element 1100 includes a first end 1104 and a second end 1106 opposite the first end 1104. In some embodiments, a lumen 1102 extends between the first and second ends 1104 and 1106. The lumen 1102 may be defined by the interior surface 1108 of the tubular element 1100. The ocular drainage system 1000 may be configured such that the interior surface 1108 of the tubular element 1100 is adapted to resist, or alternatively, promote or permit, cellular infiltration and tissue attachment thereto. In some embodiments, one or more films, membranes, stratums, sheaths, or sleeves may define the interior surface 1108 of the tubular element 1100 (and thus the lumen 1102). Similarly, one or more films, membranes, stratums, sheaths, or sleeves may be disposed about the exterior surface 1110 and define the exterior surface of the ocular drainage system 1000. In some embodiments, one of the first and second ends 1104 and 1106 is insertable into the anterior chamber of the eye, and the other of the first and second ends 1104 and 1106 is insertable into or otherwise attached to a vessel, such as an episcleral vein. In other embodiments, one of the first and second ends 1104 and 1106 is insertable into or otherwise attached to the eye (e.g., into or to the sclera) near an anterior chamber of the eye, and the other of the first and second ends 1104 and 1106 is insertable into or otherwise attached to a vessel. In such embodiments, the end insertable into or otherwise attached to the eye near an anterior chamber of the eye does not penetrate into the anterior chamber, but rather is configured to accept aqueous humor diffusing out of the anterior chamber through tissues of the eye. Whether inserted directly into the anterior chamber or inserted or otherwise attached to the eye near the anterior chamber, the compliant fluid conduit is configured to allow fluid egress from within an anterior chamber of a patient's eye when implanted.

The ocular drainage system 1000 is configured to promote and/or permit cellular infiltration and tissue attachment (also referred to herein as tissue ingrowth) to one or more portions of the ocular drainage system 1000. Although cellular infiltration and tissue attachment are also referred to herein as tissue ingrowth, it should be appreciated that the ocular drainage system 1000 is configured to promote and/or permit one, or the other, or both. Thus, the term tissue ingrowth should not be limited in every instance to cellular infiltration, or tissue attachment, or a combination of cellular infiltration and tissue attachment. In some embodiments, the entirety of the exterior surface 1110 of the tubular element 1100 may be configured promote or permit cellular infiltration and tissue attachment thereto. In other embodiments, cellular infiltration and tissue attachment may be promoted or permitted at one or more discrete locations along the exterior surface 1110 of the tubular element 1100, but may be resisted at one or more second portions (e.g., one or more other locations) of the exterior surface 1110 of the tubular element 1100. In some embodiments, the film, membrane, stratum, sheath, and/or sleeve forming part or all of the exterior surface 1110 may be configured promote or permit cellular infiltration and tissue attachment thereto, either on the entirety of the exterior surface 1110 or at discrete locations on the exterior surface 1110. Such configurations provide that tissue ingrowth is encouraged along one or more regions or portions of the ocular drainage system 1000. And, as discussed above, the ingrowth of tissue into the one or more regions or portions of the exterior of the ocular drainage system 1000 can help minimize micro-movement between the ocular drainage system 1000 and the surrounding tissue after it is implanted, which can help with biointegration of the ocular drainage system 1000 into the anatomy (e.g., the eye).

The tubular element 1100 may be formed from a variety of biocompatible materials, including, but not limited to, silicone, expanded polytetrafluoroethylene (ePTFE), polycarbonate, polyethylene, polyurethane, polysulfone, polyvinylidene fluorine (PVDF), polyhexafluoropropylene (PHFP), perfluoroalkoxy polymer (PFA), polyolefin, fluorinated ethylene propylene (FEP), acrylic copolymers and polytetrafluoroethylene (PTFE). The material(s) forming the tubular element 1100 may be elastic or inelastic.

In some embodiments, the tubular element 1100 is formed by a tubular melt extrusion process, and may be drawn down to a final target dimension. In other embodiments, the tubular element 1100 is formed by a tube paste-extrusion and expansion process commensurate with producing a desired wall thickness, porosity, stiffness, and/or dimension. In some other embodiments, the tubular element 1100 is formed by successively dip-coating a material onto a properly-sized mandrel followed by solvent removal and mandrel extraction processes.

In some embodiments, the tubular element 1100 is formed by one or more tape wrapping processes involving one or more tapes of the desired material (e.g. ePTFE). For example, the tape may be wrapped around a mandrel of a desired dimension and cross-section. In some embodiments, the tape is helically wound around the mandrel. The tape may be helically wound such that adjacent or successive windings overlap (completely or partially) one another, do not overlap, or some combination thereof, to achieve a tubular element having desired properties. The tape may be wrapped or wound around the mandrel one or more times to form one or more overlapping layers. One or more additional wrapping of one or more different material(s), such as similar materials possessing different microstructures, may be subsequently applied thereto (e.g., by additional windings, such as helical windings, dip-coatings, or other known methods), to achieve a tubular element having desired properties. In some embodiments, the tape windings and/or layers may be bonded together using one or more thermal or adhesive methods either before or after removal from the mandrel.

It is to be appreciated that the tubular element 1100 can be formed with a plurality of layers, where the different layers possess different physical properties, including, but not limited to, differing porosities, durometers, thicknesses, and/or wettability. For instance, in some embodiments, an outer wound layer of the tubular element 1100 may be more porous (or may include a more open microstructure) relative an inner wound layer of the tubular element 1100. As a result of the configurations of windings of layers, the ocular drainage system 1000 can be selectively configured to permit or promote cellular infiltration and tissue attachment at one or more portions or regions thereof, while resisting cellular infiltration and tissue attachment at one or more other portions or regions thereof.

For example, one or more portions or regions may include pores defined by interstices, perforations, and/or channels formed in the exterior surface 1110 that are sized and/or shaped to promote or permit tissue ingrowth, cellular infiltration, and/or tissue attachment. Interstices, perforations, and/or channels of the tubular element 1100 may be naturally occurring, or may be formed artificially. For instance, in some embodiments, one or more perforation processes, such as drilling, die-punching, needle-puncturing, or laser cutting processes, may additionally or alternatively be utilized to form a plurality of perforations in one or more portions of the tubular element 1100. In some embodiments, such perforation processes may be performed before and/or after the tubular element 1100 is formed.

In some embodiments, the tubular element 1100 may include pores that have an average size from between twenty (20) microns and one hundred (100) microns, such as, for example, between forty (40) and eighty (80) microns. In other embodiments, the average size of the pores may exceed one hundred (100) or one hundred fifty (150) microns. In some embodiments, the pore size corresponds to the fibril length and nodal spacing in an expanded polytetrafluoroethylene (ePTFE) microstructure. In other embodiments, the pore size may correspond to a weave pattern (e.g., in woven or knit materials). In yet other embodiments, the pore size may correspond to the arrangement of fibers (e.g., in electro-spun constructions where a polymer is dissolved in solvent and the solution is then delivered to a mandrel to successively build-up a fibrous material layer).

In some embodiments, one or more portions or regions (e.g., the interior surface 1108) of the tubular element 1100 may be non-porous (e.g., pore-free), or may include pores defined by interstices, perforations, and/or channels formed in the exterior surface 1110 that are sized and shaped to resist tissue ingrowth, cellular infiltration, and/or tissue attachment. In some embodiments, portions (or the entirety) of the tubular element 1100 include pores that are have an average size of less than one (1) or two (2) microns. Pore sizes of less than about one (1) or two (2) microns generally inhibit ingrowth of vessels and other tissue. These regions or portions of the tubular element 1100 that are configured to resist cellular infiltration may also be the result of one or more coatings or other surface treatment applications. In some embodiments, these one or more portions or regions may be rendered essentially non-porous to minimize, impede or otherwise resist tissue ingrowth.

Material coating processes may also be utilized to apply one or more drug or antimicrobial coatings (such as metallic salts (e.g., silver carbonate)) to the polymer material, and organic compounds (e.g. chlorhexidine diacetate), to the polymer material. Hydrophilic coatings to enable immediate wetout of a polymer matrix of the polymer material can also be applied as some polymer surfaces are hydrophobic in nature. Surface coatings including antioxidant components can be applied to mitigate the body's inflammatory response that naturally occurs during wound healing after surgery. Surfaces of the polymer materials can be modified with anti-proliferative compounds (e.g. Mitomycin C and 5-fluoracil), to moderate the surrounding tissue response in the eye. In some embodiments, one or more surface pre-conditioning processes may additionally or alternatively be utilized to form layers exhibiting a specific microstructure (e.g., wrinkles, folds, or other geometric out-of-plane structures), as explained in U.S. Pat. No. 9,849,629, filed Aug. 21, 2014 to Zaggl. Such surface pre-conditioning could facilitate a bolder early inflammatory phase after surgery, providing an early stable interface between the implantable device and the surrounding tissue. In some embodiments, a heparin coating may additionally or alternatively be applied to help minimize cell formation including fibrinogen buildup following implantation of the ocular drainage system 1000.

The diameter of lumen 1102 of the tubular element 1100 is of a sufficient size to facilitate flow of aqueous humor (an ocular fluid) through the ocular drainage system 1000, while avoiding an exterior diameter that significantly interferes with or impairs normal eye functions (e.g., does not interfere with blinking or regular eye movement). In some embodiments, the exterior diameter of the tubular element 1100 may range between fifty (50) and three hundred (300) microns, such as between one hundred (100) and two hundred (200) microns, although a variety of dimensions are contemplated.

The diameter of the lumen 1102 (e.g., a diameter of an internal surface of the tubular element 1100) may be constant or it may vary along a length of the tubular element 1100. For example, the tubular element 1100 may have a first diameter at a first end of the tubular element, a second diameter at a second end of the tubular element 1100, and a third diameter at a location along the length of the tubular element 1100 between the first end and the second. In this example, it is to be appreciated that the second diameter may be larger than the first and third diameters, where the third diameter may be larger than (or alternatively smaller than) the first diameter. Thus, in some embodiments, the first and second diameters may be larger than the third diameter. Alternatively, the third diameter may be larger than each of the first and second diameters. Where the diameter of the lumen 1102 varies along the length of the tubular element 1100, the diameter may vary in a continuous manner or in a discrete (e.g., stepped) manner. Additionally or alternatively, the thickness of the wall of the tubular element 1100 may be constant or may vary along the length of the tubular element 1100. Accordingly, in some embodiments, the diameter of the lumen 1102 may vary along the length of the tubular element 1100 and the wall thickness of the tubular element 1100 may vary along the length of the tubular element 1100 such that the tubular element 1100 maintains a constant outer diameter (e.g., a diameter of an exterior surface of the tubular element 1100) along its length. Alternatively, in some embodiments, the diameter of the lumen 1102 may remain constant along the length of the tubular element 1100 and the wall thickness of the tubular element 1100 may vary along the length of the tubular element 1100 such that the tubular element 1100 maintains a constant inner diameter along its length. Although examples of tubular diameter and thickness have been provided, a variety of dimensions are contemplated and are considered to be within the purview of the invention.

The length of the tubular element 1100 generally corresponds to the patient's anatomy (e.g., twenty-five (25) millimeters) and may be pre-selected from a kit that includes a plurality of differently sized tubular elements, for example, or may be formed by modifying a tubular element 1100 having a generic length. Thus, in some embodiments, the tubular element 1100 may exceed the actual length of the patient's anatomy, in which case physicians can trim the tubular element 1100 to the desired/required length needed either prior to or during the implantation procedure.

In some embodiments, the tubular element 1100 is compliant, forming a compliant fluid conduit. Compliance is generally considered to be the inverse of stiffness and can be understood to represent the tolerance of a material to undergo deformation or distortion when subjected to stress. Compliant materials are understood to have a low elastic modulus, and thus minor stress can result in considerable strain (also referred to a low modulus of elasticity). By comparison, a stiff material (or a relatively non-compliant material) does not deform much when subjected to stress (also referred to as exhibiting a high modulus of elasticity). Thus, in some embodiments, the tubular element 1100 may be compliant in that it is configured to undergo deformation or distortion when subjected to relatively minor stress. Deformation may be in the form of radial and/or longitudinal compliance.

In some embodiments, the compliance of the tubular element 1100 can be characterized by a lack of structural integrity corresponding to the tubular element 1100 losing a significant portion of its cross-sectional area when the tubular element 1100 is required to support its own weight and, with the exception of gravity, no other external forces act upon the tubular element 1100. Additionally or alternatively, the compliance of the tubular element 1100 can be characterized by its bending stiffness.

The tubular element 1100 may thus be understood to generally lack the structural integrity (e.g., hoop strength and/or column strength) required to avoid collapsing under its own weight and/or to be advanceable within the anatomy without some form of temporary structural support to aid during implantation or advancement within the anatomy. In some embodiments, support is provided to the tubular element 1100 by a separate support component (e.g., a mandrel extending within the tubular element 1100 or exterior thereto) as described below.

In various embodiments, the tubular element 1100 may be subjected to one or more material conditioning processes to achieve structurally sound first and/or second ends 1104 and 1106, or to enhance the structural integrity of other portions along the length of the tubular element 1100, with more compliant portions forming a remainder of the tubular element 1100. Additionally or alternatively, in some embodiments, the tubular element 1100 the material may be subjected to one or more material pre-conditioning processes such that, upon subsequent construction of the tubular element 1100, one or more of the first and second ends 1104 and 1106 are sufficiently structurally sound in accordance with the discussion herein.

In some embodiments, to avoid a potential risk that aqueous humor in the anterior chamber is restricted from entering the lumen 1102 of the tubular element 1100, the tubular element 1100 may be configured such that one or more of the first end 1104 and the second end 1106 is operable to maintain luminal integrity at the first and/or second ends 1104 and 1106 despite other portions of the tubular element 1100 extending therebetween lacking a sufficient amount of structural integrity to maintain luminal integrity in those regions. For instance, in some embodiments, the tubular element 1100 is configured such that an end of the tubular element 1100 (e.g., first end 1104) that is positioned within the anterior chamber (AC) is configured to maintain lumen integrity and to avoid collapse or otherwise significant deformation of the lumen 1102 at least in the region proximate that end (e.g., first end 1104). Complications associated with micro-movement and micro-irritation due to rigidity are generally avoided because the relatively more structurally sound end (e.g., first end 1104) of the tubular element 1100 is suspended within the aqueous humor of the anterior chamber (AC), and thus does not interact with tissue in a manner that could lead to micro-irritation.

Additionally or alternatively, one or more structural members, such as one or more stents, struts, and/or reinforcing elements may be incorporated, integrated, or coupled to one or more of the first and second ends 1104 and 1106 to achieve a tubular element 1100 having sufficiently structurally sound first and/or second ends 1104 and 1106. Such stents, struts, and/or reinforcing elements may be formed of any suitable biocompatible material (e.g., natural materials, or synthetic materials such as metals and polymers) discussed herein. In some embodiments, one or more of the first and second ends 1104 and 1104 of the tubular element 1100 may be flared. In some embodiments, a localized densification to the first and second ends 1104 and 1106 of the tubular element 1100, or other portions of the tubular element 1100, can increase a structural integrity thereof to an extent sufficient to resist closure forces exerted thereon by the body tissue. In some embodiments, one or more of the first and second ends 1104 and 1106 (or end portions) may be selectively coated or imbibed with a material such that the first end 1104 and/or second end 1106 has an increased resiliency, or an increased hoop strength relative to the portion of the tubular element 1100 situated between the first and second ends 1104 and 1106. For example, one or more of the first and second ends 1104 and 1106 of the tubular element 1100 may be selectively imbibed with silicone or another suitable material. Increasing the resiliency and/or hoop strength of one or more of the first and second ends 1104 and 1106 relative to the portion of the tubular element 1100 situated between the first and second ends 1104 and 1106 may be done to help increase the structural integrity of the first end 1104 and/or the second end 1106 which can help avoid collapse or failure of the first end 1104 and/or the second end 1106 due to the forces exerted on the first end 1104 and/or the second end 1106 by the patient's anatomy.

In some embodiments, where a compliant tube is not desired, one or more stents, struts, and/or reinforcing elements may be incorporated, integrated, or coupled to the tubular element 1100 in addition to or in lieu of incorporation of the same into the first and/or second ends 1104 and 1106. In some embodiments, densification of the tubular element 1100 in addition to or in lieu of a localized densification to the first and second ends 1104 and 1106 of the tubular element 1100 may be performed to increase a structural integrity of the tubular element 1100 to an extent sufficient to resist closure forces exerted thereon by the body tissue. In some embodiments, in addition to or in lieu of selectively coating or imbibing the first end 1104 and/or second end 1106 with silicone (e.g., silicone rubber), the portion of the tubular element 1100 between the first end 1104 and the second end 1106 may additionally or alternatively be selectively coated or imbibed with a material (e.g., silicone rubber) to increase resiliency and/or hoop strength of the same.

In various embodiments, the tubular element 1100 may be porous (micro- or macro-porous) or non-porous, or may include a combination of porous (micro- or macro-porous) portions and non-porous portions. In some embodiments, the tubular element 1100 may have a length defined by a first portion and a second portion. In some embodiments, the first portion may be a non-porous portion while the second portion is a porous portion. In some embodiments, the non-porous portion is impermeable to ocular fluid (e.g., aqueous humor) and resistant to tissue ingrowth while the porous portion is permeable to ocular fluid. The porous portion may be configured to resist or permit tissue ingrowth while remaining permeable to ocular fluid. Thus, in some embodiments, ocular fluid evacuated from the chamber of the eye by the tubular element 1100 may subsequently percolate through the porous portion of the tubular element 1100 to the surrounding tissue at a desired rate. The porosity (micro- or macro-) of the porous portion of the tubular element 1100 will determine the rate at which aqueous humor percolates through the porous portion. For instance, ocular fluid will percolate through a micro-porous portion at a slower rate than will ocular fluid percolate through a macro-porous portion.

In some embodiments, the portion of the tubular element 1100 configured to extend within the chamber of the eye may have an outer surface that is impermeable to ocular fluid or cellular infiltration, while the portion of the outer surface of the tubular element 1100 extending outside of the chamber (e.g., between the anterior chamber and the vessel in which the tubular element 1100 terminates) may be configured to promote or otherwise permit tissue or cellular ingrowth penetration and/or may be permeable to ocular fluid. In some embodiments, an inner surface of the tubular element 1100 may be impermeable to ocular fluid and configured to minimize the tissue ingrowth. In some embodiments, the porosity of the tubular element 1100 may vary along a length of the tubular element 1100. Additionally or alternatively, in some embodiments, the porosity of the tubular element 1100 may vary radially through a tubular wall of the tubular element 1100, which operates to control a depth to which ingrowth can occur. In some embodiments, one or more of the first and second ends 1104 and 1106 (or end portions) may be selectively coated or imbibed with a material such that the first end 1104 and/or second end 1106 has a decreased permeability relative to the portion of the tubular element 1100 situated between the first and second ends 1104 and 1106. For example, one or more of the first and second ends 1104 and 1106 of the tubular element 1100 may be selectively imbibed with silicone or another suitable material.

The flow of ocular fluid (e.g., aqueous humor) through the ocular drainage system 1000 is generally governed by a pressure difference across the tubular element 1100, which is a function of a pressure differential between opposing ends of the tubular element 1100 and a resistance to flow through the tubular element. In instances where the ocular drainage system 1000 includes a first end 1104 disposed within the anterior chamber (AC) and a second end 1106 disposed within a vessel, the pressure difference across the tubular element 1100 may thus be a function of the pressure differential between the intraocular pressure within the anterior chamber and the pressure within the vessel, as well as the resistance to flow of aqueous humor through the lumen 1102 of the tubular element 1100.

Flow resistance through a tube is a function of tubular element flux resistance (e.g., based on tube geometry, diameter, length, and the Hagen-Poiseuille Equation). As mentioned above, however, the tubular element 1100 is generally soft and compliant, exhibits low column and hoop strength, and is generally incapable of supporting its own weight. That is, in some embodiments, the tubular element 1100 lacks a sufficient amount of structural integrity necessary to avoid collapsing under its own weight. Accordingly, flow through the tubular element 1100 is further dependent upon the force required to maintain an inflation (partial or total) of the lumen 1102 of the tubular element 1100.

In some embodiments, the intraocular pressure of the anterior chamber inflates or otherwise operates to maintain the generally tubular geometry and avoid collapse of the lumen 1102 of the tubular element 1100. That is, in some embodiments, the aqueous humor flowing through the lumen 1102 of the tubular element 1100 operates to inflate the lumen 1102. Thus, despite being soft and compliant, the tubular element 1100 is sufficient to operate as a conduit for evacuating aqueous humor from the anterior chamber of an eye, under appropriate conditions, such as, for example increased intraocular pressure requiring implantation of the ocular drainage system 1000. In various embodiments, because the tubular element 1100 is soft and compliant, it is operable to conform to the curvature of the eye and avoid interfering with normal eye function (e.g., pivoting and blinking).

Turning now to FIG. 5, an ocular drainage system 1000 is shown that includes a tubular element 1100 consistent with the tubular element 1100 discussed above, as well as a sheath 1200 disposed about the tubular element 1100. The sheath 1200 is configured to promote or permit cellular infiltration and tissue attachment consistent with the discussion above regarding cellular infiltration and tissue attachment in the tubular element 1100. That is, like the tubular element 1100, the sheath 1200 may be formed of a biocompatible material that includes a plurality of pores that are sized to promote and/or permit cellular infiltration and tissue attachment. In some embodiments, the sheath 1200 may include a microstructure exhibiting cellular infiltration and tissue attachment promotive/permissive properties. Alternatively, the sheath 1200 may be configured to resist cellular infiltration and tissue attachment.

In some embodiments, the sheath 1200 may include one or more layers or sheets of expanded polytetrafluoroethylene (ePTFE). However, these layers or sheets may be additionally or alternatively formed from other polymers, including, but not limited to, polyurethane, polysulfone, polyvinylidene fluorine (PVDF), polyhexafluoropropylene (PHFP), perfluoroalkoxy polymer (PFA), polyolefin, fluorinated ethylene propylene (FEP), acrylic copolymers, and polytetrafluoroethylene (PTFE). These materials can be in sheet, knitted, woven, or non-woven forms. In some embodiments, the sheath 1200 is formed from a plurality of layers or sheets of polymer material. In some such embodiments, the layers or sheets are laminated or otherwise mechanically coupled together, such as by way of heat treatment and/or high-pressure compression and/or adhesives and/or other laminating methods known by those of skill in the art.

In some embodiments, the layers or sheets of polymer material forming the sheath 1200 is subjected to one or more processes to modify the microstructure (and thus the material properties) of the layered polymer material. In some embodiments, such processes include but are not limited to, material coating processes, surface pre-conditioning processes, and/or perforation processes consistent with the discussion above.

The sheath 1200 may be configured such that it is permeable to ocular fluid (e.g., aqueous humor), or more permeable to ocular fluid by way of a perforation process and/or by way of a naturally occurring microstructure of the polymer(s) forming the sheath 1200. In some embodiments, the sheath 1200 may include pores that have an average size that is between about twenty (20) microns and about one hundred (100) microns, or between about forty (40) and about eighty (80) microns. In other embodiments, the size (or average size) of the pores may exceed one hundred (100) or one hundred fifty (150) microns.

In some embodiments, after placing one of the first and second ends 1104 of the tubular element 1100 into the anterior chamber, the tubular element 1100 may be fastened to the surrounding tissue to help minimize a risk of dislodgement of the tubular element 1100 from within the anterior chamber. In some embodiments, one or more stitches are utilized to couple the tubular element 1100 (and thus the ocular drainage system 1000) to the eye tissue. In some embodiments, a biocompatible tissue adhesive may be used. In other embodiments, a needle track created through the tissue to provide access to the anterior chamber in association with the implantation of the tubular element may be sufficiently sized such as to provide an interface fit capable of retaining the tubular element 1100.

It is to be appreciated that while the tubular element 1100 illustrated and described herein includes a generally circular cross-section, the tubular element 1100 may have a cross-section of any suitable shape without departing from the spirit or scope of the disclosure. For instance, the tubular element 1100 may include a cross-section that is ovular, square, rectangular, trapezoidal, or any other polygonal shape such that it does not interfere with normal eye function.

In some embodiments, the ocular drainage system 1000 may further include a stiffening member that is removably integrated with the tubular element 1100. The stiffening member (not shown) helps aid in the delivery of the tubular element 1100, as the soft and compliant nature of the tubular element 1100 makes advancement thereof through the anatomy difficult. Accordingly, the removable stiffening member operates with the tubular element 1100 to temporarily form an installation assembly having column strength in excess of the column strength of the tubular element 1100. Forming an installation assembly having such an increased column strength helps aid in the delivery/implantation of the ocular drainage system 1000. Such an installation assembly also helps provide for delivery of an ocular drainage system 1000 that is compliant and operable to conform to the tissue (e.g., eye tissue) and profile of the anatomy in which the ocular drainage system 1000 is being implanted, while maintaining a minimum profile to avoid irritation and/or interference with normal body functions (e.g., blinking of the eye).

The stiffening member, which has sufficient column strength to enable its advancement within the anatomy, helps enable advancement of the tubular element 1100 within the anatomy. Once properly positioned within the anatomy, the stiffening member can be removed from the tubular element 1100 in situ without also requiring removal of the tubular element 1100 from its position within the anatomy. In some embodiments, the stiffening member may be an elongate element situated within an interior of and/or about an exterior of the tubular element 1100. The elongate element may be coiled or may extend longitudinally (e.g., in the form of a mandrel). For example, the stiffening member may be in the form of a coiled suture.

The stiffening member may include silicone, ePTFE, polycarbonate, polyethylene, polyurethane, polysulfone, PVDF, PHFP, PFA, polyolefin, FEP, acrylic copolymers and other suitable fluoro-copolymers, or any other suitable polymer, or metallic components such as stainless steel or nitinol (straight or braided). It will be appreciated that the material properties of the stiffening member and/or gauge can be varied to produce stiffening members of a desired axial, lateral, and/or radial stiffness. In other embodiments, the stiffening member may additionally or alternatively be formed of an ablateable or an absorbable material. The ocular drainage system 1000 may be formed with the stiffening member removably coupled with the tubular element 1100, or the stiffening member may be inserted into the tubular element 1100 by the user prior to implantation. The stiffening member may then be removed or decoupled from the tubular element 1100 after implantation.

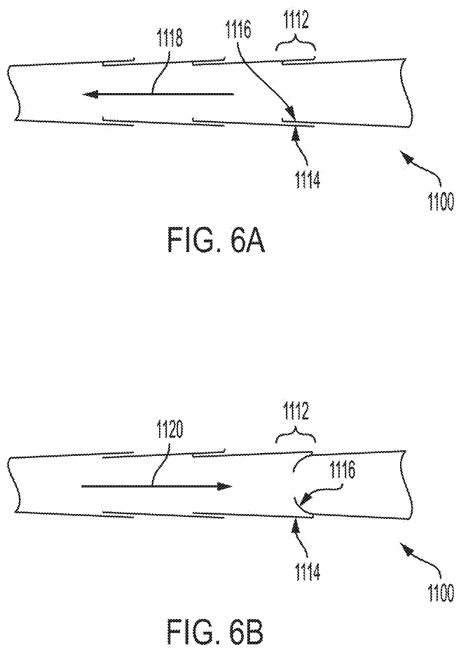

Turning now to FIGS. 6A and 6B, detailed views of a section of a tubular element 1100 of an ocular drainage system 1000 are shown. The tubular element 1100 shown in FIGS. 6A and 6B includes a helically wrapped construct where adjacent helical wraps overlap in overlap regions 1112. Although the amount of overlap in each of the overlap regions 1112 is depicted to be the same, it is to be appreciated that the amount of overlap may be varied. For instance, an amount of overlap may be varied to modify properties (e.g., structural integrity) along different portions of the tubular element 1100. The overlap regions generally include an inner layer and an outer layer, such as outer and inner layers 1114 and 1116 shown in FIGS. 6A and 6B. Adjacent helical wraps are generally bonded together along the overlap regions such that the adjacent helical wraps are bonded along an interface between respective inner and outer layers, with the exception of one or more discrete regions, where a portion of the interface between the inner and outer layers of adjacent helical wraps remains unbonded. That is, only a portion of the inner layer 1116 in the overlapping region is bonded to the outer layer 1114. For example, only a portion of the inner layer 1116 in the overlap region 1112 is bonded to the outer layer 1114. Such a configuration provides that, in the designated unbonded region, the inner layer 1116 can deflect radially inwardly away from the outer layer 1114 (as shown in FIG. 6B) to operate as a restriction (or valve) in response to fluid flowing through the tubular element 1100 in an undesirable direction (e.g., reverse flow). That is, in various embodiments, the inner layer 1116 is configured to deflect radially inwardly away from the outer layer 1114 under reverse flow conditions to reduce the diameter of the tubular element 1100 proximate or at the overlap region 1112 to reduce, minimize, or obstruct the reverse flow.

In various embodiments, the tubular element 1100 is formed by helically wrapping material to form overlapped regions such that an exposed leading edge of the inner portions of the overlapping regions face downstream under normal operating conditions (e.g., where ocular fluid is flowing through the tubular element 1100 in the direction of arrow 1118), as shown in FIG. 6A. That is, the material forming the tubular element 1100 is helically wrapped such that the inner layer 1116 extends beneath the outer layer 1114 in the direction of the flow of ocular fluid under normal operating conditions (e.g., aqueous humor flowing along arrow 1118).

FIG. 6A shows the tubular element 1100 under normal flow operation where aqueous humor is flowing out of the anterior chamber (AC) of the eye, and through the tubular element 1100 away from the anterior chamber (AC). Conversely, FIG. 6B shows the tubular element 1100 under abnormal flow operation where aqueous humor is backflowing through the tubular element 1100 toward the anterior chamber (AC) of the eye in the direction of arrow 1120. As shown in FIG. 6B, when aqueous humor backflows through the tubular element 1100 in the direction of arrow 1120, toward the anterior chamber (AC) of the eye, the unbonded portions of the inner layer 1116 deflect radially inwardly away from the outer layer 1114 as shown. In turn these deflected, unbonded portions of the inner layer 1116 operate to help prevent, reduce, or otherwise minimize a flow rate of aqueous humor backflowing (or reverse flowing) through the tubular element 1100 and back into the anterior chamber (AC) of the patient's eye.

In some embodiments, the ocular drainage system 1000 is implantable ab-externally (e.g., from outside of the eye), such as through a conjunctival incision. In some embodiments, a conjunctival radial incision is performed typically near the limbal junction, and blunt dissection of the conjunctiva is performed to expose the sclera and provide a site for placement of ocular drainage system 1000. In some embodiments, this may require suturing one or more portions of the ocular drainage system (e.g., the tubular element 1100) to the sclera. In some embodiments, a small needle, such as a 22 or 23 gauge needle, is also inserted near the scleral spur to provide a track for subsequent insertion and placement of the tubular element 1100 into the anterior chamber.

In various embodiments, one or more portions of the ocular drainage system 1000 may include or be coated by one or more therapeutic agents such as medications that treat glaucoma.

In some embodiments, the ocular drainage system 1000 includes one or more erosion elements (not shown) extending adjacent a portion of the tubular element 1100 that helps minimize the potential for erosion of the tubular element 1100 through tissues of the eye when the ocular drainage system 1000 is implanted. In some embodiments, the erosion element is a plate that is situated between the tubular element 1100 and the surrounding tissue such that any micro-movement of the tubular element 1100 occurs between the tubular element 1100 and the erosion element while the erosion element remains stationary relative to the eye tissue. Thus, the erosion element operates as a protective barrier between the tubular element 1100 and the tissue. The erosion element may be integral to the tubular element 1100 or may be coupled thereto via one or more fastening elements, such as one or more sutures, adhesives, or the like. In some embodiments, the erosion element may include any material discussed herein as being suitable for the tubular element 1100.

In some embodiments, the tubular element 1100 of the ocular drainage system 1000 may be initially partially or entirely obstructed with one or more resistive elements (not shown) that are removably situated in the lumen or lumens of the tubular element 1100, such that fluid flow through the tubular element 1100 is initially blocked or reduced by the resistive elements. These resistive elements can be later removed from the tubular element 1100 to allow for an increased flow rate of fluid through the tubular element 1100 relative to the flow rate through the tubular element 1100 prior to removal of the resistive element.

In some embodiments, the resistive elements may be bioabsorbable such that they are configured to bio-disintegrate over time, such as by way of interacting with bodily fluids (e.g., aqueous humor). Additionally or alternatively, the resistive element can be configured to be removed by way of some physical intervention, including physical retrieval by a physician, and/or by way of ablation by a high energy source (e.g., a laser). In embodiments where the tubular element 1100 may include a plurality of lumens (e.g., where the tubular element is formed with a plurality of lumens or where the tubular element is formed of a plurality of individual elements having lumens therethrough that collectively form the tubular element 1100), a resistive element may be initially positioned within one or more of the plurality of lumens such that flow through the lumen is blocked or reduced, thereby reducing flow rate through the tubular element 1100 relative to a flow rate through the tubular element 1100 when the resistive elements are removed from the lumen or lumens.

In certain embodiments, the ocular drainage system 1000 may be implanted to reduce intraocular pressure caused by excessive ocular fluid build-up. The ocular drainage system allows for excess ocular fluid to drain from a chamber of the eye and be reabsorbed. In particular embodiments, the ocular drainage system 1000 may be implanted to reduce the symptoms of or treat ocular hypertension or glaucoma. In such embodiments, the ocular drainage system 1000 may be configured to drain aqueous humor from an anterior chamber of a patient's eye.

### EXAMPLE 1

Firstly, an ePTFE film of about 0.150″ in width was helically wrapped over a center wire of silver-plated-copper of about 0.010″ in diameter, resulting in a coverage of about 5 layers of ePTFE. The wrapped construct was then subjected to a thermal treatment in a convective air oven at 360 C for about 10 minutes. Once cooled, the center wire was drawn and removed, leaving a patent ePTFE tube.

Secondly, a small batch of 2-part silicone (Nusil Inc., Grade 4840 Carpinteria, Calif. 93013) was prepared resulting in a thick viscous fluid. This fluid was diluted 50% with n-heptane and mixed thoroughly.

Thirdly, using a syringe affixed with a 25-gauge needle, the silicone fluid was injected into the ePTFE tube. A 0.010″ straight wire was then inserted into the fluid filled tube. The assembly was then subjected to a 115 C thermal treatment for about 15 minutes. Once cooled, the straight wire was removed leaving a patent, water-tight ePTFE tube with an exterior configured to permit tissue ingrowth.

### EXAMPLE 2

Firstly, an ePTFE film of about 0.070″ in width was helically wrapped over a center wire of silver-plated-copper of about 0.005″ in diameter, resulting in a coverage of about 2 layers of ePTFE. The wrapped construct was then subjected to a thermal treatment in a convective air oven at 360 C for about 10 minutes.

Secondly, a small batch of 2-part silicone (Nusil Inc., Grade 4840 Carpinteria, Calif. 93013) was prepared resulting in a thick viscous fluid. This fluid is diluted approximately 50% with n-heptane (Item 246654, Sigma-Aldrich Corp. St. Louis, Mo.) and mixed thoroughly.

Thirdly, the wrapped mandrel was then held in tension horizontally and was coated with an excess of the viscous silicone mixture. The coated wrapped mandrel was then run through pinched thumb and index-finger to meter the excess silicone though still providing a coated exterior surface.

Fourthly, an ePTFE film of about 0.070″ in width was helically wrapped over the coated wrapped mandrel. This assembly was then subjected to a thermal treatment of 115 C for 15 minutes. Once cooled, the straight wire was removed leaving a patent, water-tight ePTFE tube with an exterior configured to permit tissue ingrowth.

The inventive scope of this application has been described above both generically and with regard to specific examples. It will be apparent to those skilled in the art that various modifications and variations can be made in the examples without departing from the scope of the disclosure. Likewise, the various components discussed in the examples discussed herein are combinable. Thus, it is intended that the examples cover the modifications and variations of the inventive scope.

1. A medical device including:
a compliant fluid conduit configured for implantation within a biological tissue, the compliant fluid conduit having a first end, a second end, a lumen, and an exterior having a microstructure that is configured to permit cellular ingrowth therein;
where the first end is configured to be inserted into an eye of a patient to allow ocular fluid to drain from the eye; and
where the second end is configured to be inserted into an ocular venous system of the patient to allow the ocular fluid drained from the eye to flow directly into the ocular venous system.

2. The device of claim 1, where a luminal wall surface of the lumen is configured to resist cellular ingrowth and attachment.

3. The device of claim 2, where the luminal wall surface of the lumen includes a plurality of pores sized to resist cellular ingrowth and attachment.

4. The device of claim 2, where the luminal wall surface of the lumen includes a microstructure that is configured to resist cellular ingrowth and attachment.

5. The device of claim 1, where the compliant fluid conduit is a polymer tube.

6. The device of claim 5, where the polymer tube includes a plurality of layers.

7. The device of claim 6, wherein the plurality of layers include a first layer having a first micro-structure and a second layer having a second micro-structure.

8. The device of claim 5, where the polymer tube comprises a fluoropolymer.

9. The device of claim 8, where the polymer tube comprises expanded polytetrafluoroethylene.

10. The device of claim 1, where the device operates to regulate an intraocular pressure of a patient's eye when implanted.

11. The device of claim 1, where the compliant fluid conduit is configured to allow fluid egress from within an anterior chamber of a patient's eye when implanted.

12. The device of claim 1, where the compliant fluid conduit comprises one of a plurality of lumens formed in a tubular structure, or a plurality of individual tubular elements, each tubular element including a lumen extending therethrough.

13. The device of claim 1, further including a valve configured to regulate a rate of fluid flowing through the compliant fluid conduit.

14. The device of claim 13, where the valve is formed from partially unbonded helical windings of a material forming the compliant fluid conduit, where the valve is configured to regulate a rate of fluid backflowing through the compliant fluid conduit in a direction toward an anterior chamber of the eye.

15. The device of claim 14, where the valve is integral to the compliant fluid conduit such that the valve and the compliant fluid conduit form a monolithic unit.

16. The device of claim 1, where the exterior of the compliant fluid conduit includes a plurality of pores sized to permit cellular ingrowth.

17. The device of claim 16, where the interior of the compliant fluid conduit includes a microstructure that is configured to resist cellular ingrowth and attachment.

18. The device of claim 1, further including a sheath disposed about the compliant fluid conduit, the sheath defining the exterior of the compliant fluid conduit.

19. The device of claim 1, where the second end of the compliant fluid conduit is configured to be inserted into an episcleral vein of the eye.

20. The device of claim 1, where the compliant fluid conduit is a synthetic polymeric material that is nonbioabsorbable.

21. A method of treating glaucoma, the method including:
providing a compliant fluid conduit having a first end configured for insertion into an eye of a patient and a second end configured for insertion into an ocular venous system of the patient, the compliant fluid conduit being configured for implantation within a biological tissue and including an exterior that is configured to permit cellular ingrowth therein;
inserting the first end into the eye of the patient such that the first end of the compliant fluid conduit accesses a fluid reservoir within the eye; and
inserting the second end of the compliant fluid conduit into the ocular venous system such that a fluid within the fluid reservoir within the eye is free to drain through the compliant fluid conduit into the ocular venous system.

22. The method of claim 21, where inserting the first end of the compliant fluid conduit into the eye of the patient includes inserting the first end into an anterior chamber of the eye of the patient.

23. The method of claim 21, where inserting the second end of the compliant fluid conduit into the ocular venous system includes inserting the second end into an episcleral vein of the eye.

24. The method of claim 21, where the compliant fluid conduit is a synthetic polymeric material that is nonbioabsorbable.
